(19) United States
(12) Patent Application Publication
(10) Pub. No.: US 2004/0230259 A1
(43) Pub. Date: Nov. 18, 2004

(54) Apparatus and method for treatment of acne

(76) Inventor: Thierry Fabio Di Matteo, Chula Vista, CA (US)

Correspondence Address: LAW OFFICE OF MICHAEL P. EDDY, #510, 2683 VIA DE LA VALLE, SUITE G, DEL MAR, CA, 92014, US

(21) Appl. No.: 10/787,867

(22) Filed: Feb. 26, 2004

Related U.S. Application Data
(60) Provisional application No. 60/450,229, filed on Feb. 26, 2003.

(51) Int. Cl. A61N 1/00

(52) U.S. Cl. 607/88

(57) ABSTRACT

The present invention relates to the treatment of dermatological conditions, more particularly acne. An inventive apparatus emits a typical peak emission frequency from wavelengths in the range of 395 nm to 450 nm or a first typical peak emission spectrum in the range of 405 nm to 415 nm and a second emission spectrum substantially in the range of 410 nm to 420 nm. Another inventive apparatus emits an additional typical peak emission frequency of 660 nm. Inventive methods include the steps of applying a topical oil solution, used for the treatment of controlling oil, to a portion of skin, removing said solution from the skin, and applying illumination from the aforementioned apparatuses to an affected portion of skin.

### CROSS-REFERENCES TO OTHER RELATED PATENT APPLICATIONS

This application claims the benefit of U.S. Provisional Application Ser. No. 60/450,229, filed 26 Feb. 2003, the contents of which are incorporated herein by reference.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND OF THE INVENTION

The present invention is an apparatus and method related to the elimination or reduction of the dermatological condition acne.

### BRIEF SUMMARY OF THE INVENTION

One inventive apparatus of the present invention for the treatment of acne comprises a plurality of light emitting diodes mounted on a substrate, the first group of diodes with either a typical peak emission frequency from wavelengths in the range of 395 nm to 450 nm or a first typical peak emission spectrum in the range of 405 nm to 415 nm and a second emission spectrum substantially in the range of 410 nm to 420 nm; wherein said plurality of light emitting diodes are operated by an electronic circuit at various frequencies in a forward voltage/current function with electronic and electromechanical components affixed in a controlling circuit and integrated with a common power source.

Another inventive portable lamp apparatus of the present invention for the treatment of acne comprises a plurality of fluorescent tubes, wherein one or more tubes emits a typical peak emission frequency with wavelengths either in the range of 395 nm to 450 nm or in the range of 405 nm to 415 nm, and one or more tubes emits a typical peak emission frequency in the range of 410 nm to 420 nm.

An inventive method of the present invention for the treatment of acne comprises the steps of applying a topical oil solution, used for the treatment of controlling oil, to a portion of skin, removing said solution from the skin, and applying illumination to said portion of skin with an apparatus comprised of a plurality of light emitting diodes mounted on a substrate, the first group of diodes with either a typical peak emission frequency from wavelengths in the range of 395 nm to 450 nm or a first typical peak emission spectrum in the range of 405 nm to 415 nm and a second emission spectruim substantially in the range of 410 nm to 420 nm; wherein said plurality of light emitting diodes are operated by an electronic circuit at various frequencies in a forward voltage/current function with electronic and electromechanical components affixed in a controlling circuit and integrated with a common power source.

A second inventive method for the treatment of acne comprises the steps of applying a topical oil solution, used for the treatment of controlling oil, to a portion of skin, removing said solution from the skin, and applying illumination to the affected portion of skin with a portable lamp apparatus comprised a plurality of fluorescent tubes, wherein one or more tubes emits a typical peak emission frequency with wavelengths either in the range of 395 nm to 450 nm or in the range of 405 nm to 415 nm, and one or more tubes emits a typical peak emission frequency in the range of 410 nm to 420 nm.

The aforementioned apparatuses, as well as the apparatuses used in conjunction with the inventive methods, can additionally be comprised of a third group of one or more light emitting diodes with a typical peak emission frequency of 660 nm.

### BRIEF DESCRIPTION OF THE SEVERAL VIEWS OF THE DRAWINGS

The above and other objects, advantages and features of the present invention will be more readily apparent from the following description, when read in conjunction with the accompanying drawings wherein:

FIG. 1 is schematic diagram of a circuit of an embodiment of the present invention;

FIG. 2 is schematic diagram of a second circuit one embodiment of the present invention;

FIG. 3 is front view of the printed circuit board assembly;

FIG. 4 is a front elevation view of a portable lamp embodiment with the lamp portion rotated to show the light emitting diodes and circuit board base;

FIG. 5 is a front elevation view of the head of a portable lamp embodiment with a lens cover attached;

FIG. 5A is a side elevation view of the head of a portable lamp embodiment;

FIG. 6 is a front elevation view of a pen style embodiment of the present invention;

FIG. 7 is a front elevation view of a battery powered hand held embodiment of the present invention;

FIG. 8 is a front elevation view of the head of a battery powered hand held lamp embodiment;

FIG. 8A is a side elevation view of the head of a battery powered hand held lamp embodiment;

FIG. 9 is a front elevation view of the head mounted embodiment of the present invention;

### DETAILED DESCRIPTION OF THE INVENTION

One object of the present invention is to provide an apparatus and method using the apparatus to aide in the alleviation or elimination of acne causing bacteria propioni-bacterium acnes (P.A.) in skin pores. Studies have shown that anaerobic bacteria such as P.A. are destroyed in skin pores through action such as where there is release and formation of oxygen or its derivatives as well as through photo-destruction.

The portable light source of the first embodiment is comprised of light emitting diodes mounted on a rigid, fixed or inflexible substrate such as a FR-4 material printed circuit board, a ceramic substrate or any equivalent. The light emitting diodes are arranged in a small, common and planar array wherein the array is comprised of a primary group of light emitting diodes having a peak emission frequencies either from wavelengths 395 nm to 450 nm or from wavelengths 405 nm to 415 nm and from wavelengths 410 nm to 420 nm. These peak emission frequencies of the diodes are selected as to produce two separate and different peaks or plateaus, such as in the blue and violet ranges, when the diodes are operated in conjunction with a lamp having an electronic circuit at various frequencies in a forward voltage/current function with electronic and electro-mechanical components affixed in a controlling circuit and integrated with a common power source.

An operation of the inventive apparatus according to the first embodiment is described hereunder. The apparatus is configured as a portable light source, which can be used for illuminating skin in a therapeutic dermatology application utilizing an appropriate and sufficient intensity which can be applied multiple times to human skin over specific time intervals. This illumination aides in the alleviation or elimination of acne causing bacteria propioni-bacterium acnes—P.A. in skin pores, through the action of or formation/release of oxygen and/or its derivatives and/or photo-destruction, thus effectively killing anaerobic bacteria such as P.A. The illumination may also alleviate or eliminate possible side effects or residual effects of the P.A. in skin pores.

In the first embodiment, the apparatus contains an array of electronically activated light emitting diodes (LEDs) so that the apparatus emits at a minimum a blue/violet light either in the typical peak wavelengths between the range of 395 and 450 nm or between the ranges of 405 and 415 nm and 410 nm to 420 nm. These wavelengths are selected to produce at least two separate and different peaks or plateaus. The apparatus is further comprised of an electronic circuit which can be operated at various frequencies and voltages, with a variety of power sources such as A.C., D.C., pulsed A.C. or pulsed D.C. Additionally, the output luminous intensities of the inventive apparatus, frequently measured in lumens per square foot or watts per centimeter, can be adjusted as desired.

The LEDs used in the inventive apparatus are commercially available through various manufacturers, for example the Panasonic “LNXXX” line of LEDs or the Lumex “SSL-LX50XXXXX” family of LEDs. Any equivalent LED capable of emitting wavelengths required by the inventive apparatus can be used. In addition, the electronics of the inventive apparatus, including the first embodiment, can be configured as desired so that the results for the specific application are achieved. For instance, assembled units may include a combination of one or more electronic components, such as integrated circuits, capacitors, resistors, transformer circuits, and power sources as well as non electronic components such as a power cord.

Additionally, achieving different desired results may necessitate varying the components of the inventive apparatus, for example in the case where maximum intensity is desired. The inventive apparatus can be constructed so that no external or internal cooling mechanism, such as water jackets, air fans, heat sink, exchangers or the like are required. The housing may also contain components such as a band-UV-filter to preclude, lessen or block the external transmission of certain low frequency medically detrimental wavelengths such as UV-C which can otherwise emanate from the apparatus.

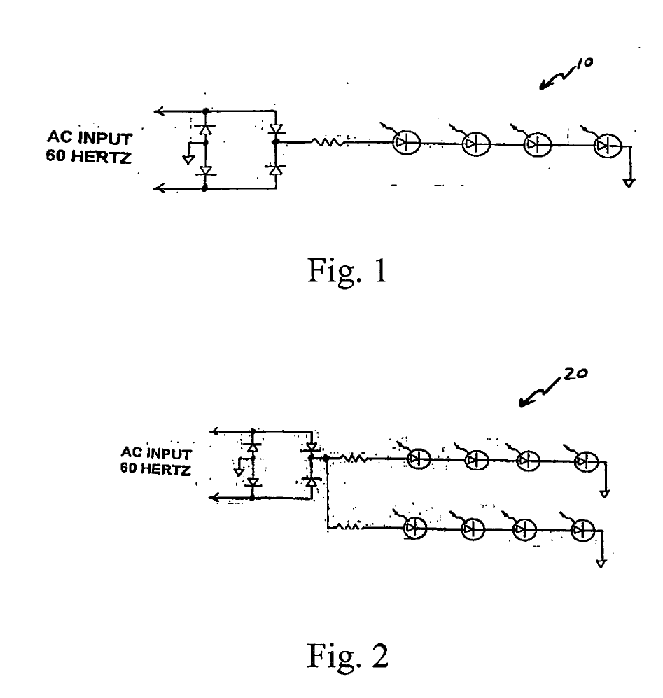

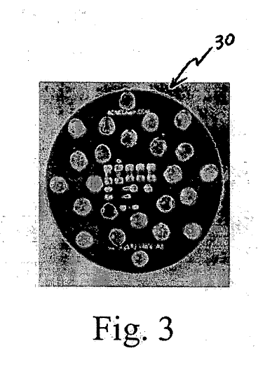

The inventive apparatus may include a separate power source or an integrated power source, which can be configured, for example, to be built into the lamp head of the first embodiment. The power source may also be incorporated into a hand held unit or a plastic or webbed head band unit to be used as a head set or the like. FIG. 1 provides an illustration of schematic circuit 10 which can emit at least two different desired typical peak wavelengths. In this embodiment, the apparatus emits two or more wavelengths between 395 nm and 450 nm. FIG. 2 shows an illustration of schematic circuit 20 which is another electronic circuit configuration. In a second embodiment, the apparatus produces one emission with a peak or plateau between 405 nm and 415 nm and another emission between 410 nm and 420 nm, with a cycle rate of 50 Hz through 10 KHz. FIG. 3 illustrates an assembly 30 comprised of a printed circuit board with a plurality of light emitting diodes disposed as to be able to emit two or more different peak wavelengths between 395 nm and 450 nm at a cycle rate in the range from 50 Hz to 10 kHz. Assembly 30 is also configurable to emit one or more typical peak wavelengths in the range of 405 nm to 415 nm and one or more typical peak wavelengths between 410 nm and 420 nm at a cycle rate in the range from 50 Hz to 10 kHz.

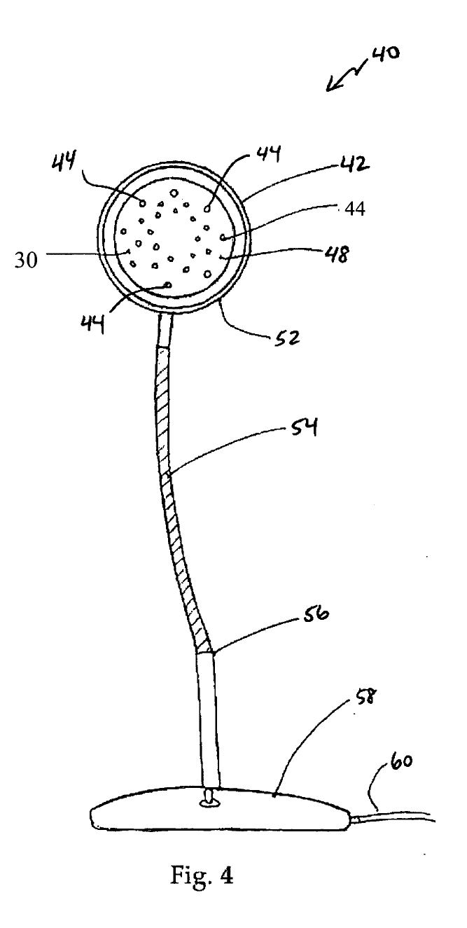

FIG. 4. is a front elevation view of a portable lamp 40 embodiment. Lamp head 42 is rotated so as to face the user with lens 48 covering printed circuit board assembly 30, including the electronic circuitry for controlling the voltage, current, frequency and the like. Also shown on printed circuit board assembly 30 is a plurality of light emitting diodes 44 situated on the surface of assembly 30 so that the light emanates away from lamp head housing 52 towards the user with the affected skin (not shown.) Portable lamp 40 also includes a goose style neck 54 for supporting the lamp head housing 52. FIG. 4 also shows that portable lamp 40 can accommodate more than one neck 54 at the base attachment position 56, which is located at the top of base 58. Additionally, power is provided to portable lamp 40 by power cord 60.

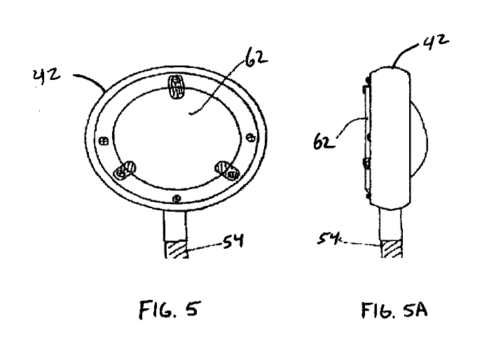

FIG. 5 is a front elevation view of the top portion of a portable lamp 40 with a lens cover 62 attached. Lens cover 62 can act as a filter or a complete block depending on the material used for the lens. The reflectance and transmittance of one or more wavelengths can be adjusted with the lens as needed for containerization and wavelength blocking. A further function of lens cover 62 is as a mechanical closure for the distal end of portable lamp 40 so the lamp 40 can be secured for storage or transport while being kept free from dirt and debris. FIG. 5A is a side view of FIG. 5.

Another embodiment of the inventive apparatus includes the ability to adjust the intensity of the emitted light when lamp 40 is activated for use. The intensity can be adjusted by the user to a low, medium, or high setting. Intensity of the emitted light may also be adjusted on the basis of a scale, such as lumens or watts per square centimeter.

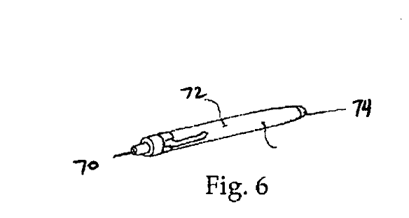

In FIG. 6, another embodiment shows the inventive apparatus as a pen style device. Power switch 70 is located at the proximal end of the pen style case 72. One or more of the light emitting diode 44 with any necessary electronics, such as a smaller variation of printed circuit board assembly 30, are accommodated in case 72. Light is emitted from case 72 towards distal end 74.

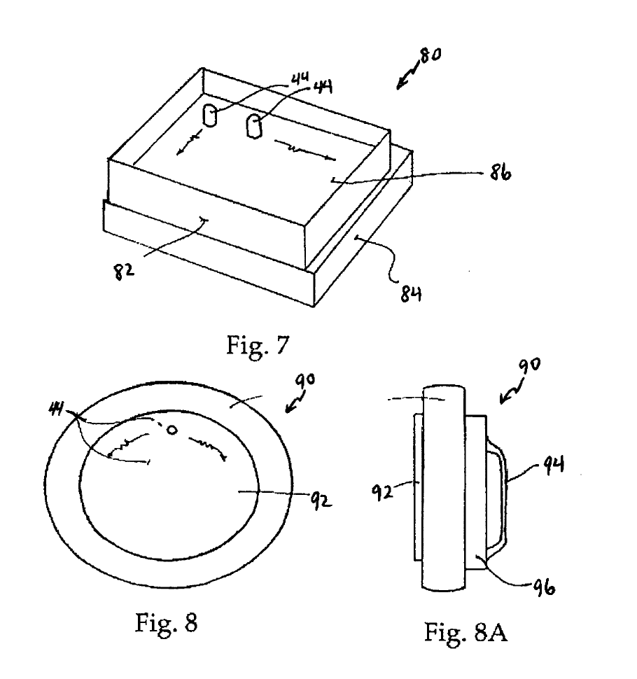

FIG. 7 is an illustration of a battery powered hand held lamp 80 accommodating a plurality of light emitting diodes 44 in a compact portable case 84. Light emitting diodes 44 are mounted on printed circuit board assembly 30 (not shown) which is connected to additional electronic circuitry required for the operation of lamp 80 and which is contained in section 82 of the present embodiment's case. Lamp 80 also accommodates one or more commercially available batteries, such as sizes AA or AAA, in case section 84, in order to provide a portable power source for lamp 80. Also not shown in lamp 80 is a on/off power switch to allow a user to turn on or off the unit, or to allow a user to turn on or off a timer integral to the unit. Lens cover 86, used optionally to cover, block or filter one or more wavelengths emitted from lamp 80, can be placed on the distal end of lamp 80.

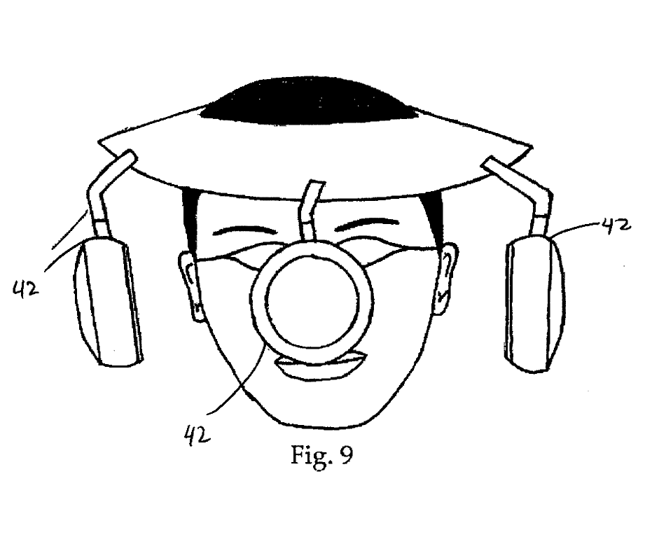

FIG. 8 is a front view a portable lamp 90 embodiment of the present invention. FIG. 8A is a side view of portable lamp 90. Lamp 90 accommodates a plurality of light emitting diodes 44 with a printed circuit board assembly 30 and any related electronics necessary for the operation of lamp 90. Lamp 90 also accommodates one or more commercially available batteries in battery case 96. A handle 94 which can also be used in conjunction with a stand is located at the rear of lamp 90. Either battery case 96 or handle 94 can be either integral to lamp 90 or separate components linked to lamp 90. Lens cover 92 is affixed to the surface of lamp 90, and can be used optionally to cover, block or filter one or more wavelengths emitted from lamp 90.

FIG. 9 is a head mounted embodiment of the present invention. The head mounted unit can be used to provide light onto a user's face area. It is able to accommodate one or more lamp heads 42, or the like, to provide illumination to an affected portion of skin.

In another embodiment of the invention (not shown,) the inventive apparatus is comprised of a portable light source using one or more fluorescent tubes in addition or instead of one or more light emitting diodes, all of which are mounted into a case sufficient to accommodate the tubes, diodes, power source and related electronic circuitry. Commercially available fluorescent tubes in sizes such as T-5 and T-8 may be used. Electronic circuitry needed to operate tubes may include one or more ballasts, mounting clips, and fluorescent tube power supplies providing power at a sufficient wattage to operate the inventive apparatus. The fluorescent tubes, or the combination of tubes and diodes, emit a peak wavelength output of 660 nm and/or from wavelengths between 395 nm and 450 nm or between wavelengths of 405 nm to 415 nm and 410 nm to 420 nm, selected as to produce two separate and different peaks or plateaus in the blue and violet ranges, which also can be produced in singular or combined multiple peak functions.

In another embodiment, the inventive method comprises the steps of first applying and removing a topical oil solution and second illuminating the affected area of the skin with an inventive light emission source such as portable lamp 40 or portable lamp 80. The topical oil solution, available commercially over the counter or by prescription, can be comprised of any available compounds or solutions used for the treatment of acne. For example, clinic O.C. gel, made by Ferndale Laboratories, Inc., and comprised substantially of purified water, propylene glycol, acrylate copolymer (DVB/isobornyl methacrylate/lauryl methacrylate copolymer,) PEG-395, carbomer 940, methylparaben, disodium EDTA and sodium Hydroxide may be used.

The embodiments described herein are illustrative and not restrictive, the scope of the invention being indicated in the appended claims and all variations which come within the meaning of the claims are intended to be embraced therein.

I claim:

1. An apparatus for the treatment of acne, the apparatus comprising:
a plurality of light emitting diodes mounted on a substrate, the first group of diodes with either a typical peak emission frequency from wavelengths in the range of 395 nm to 450 nm or a first typical peak emission spectrum in the range of 405 nm to 415 nm and a second emission spectrum substantially in the range of 410 nm to 420 nm;
wherein said plurality of light emitting diodes are operated by an electronic circuit at various frequencies in a forward voltage/current function with electronic and electromechanical components affixed in a controlling circuit and integrated with a common power source.

2. An apparatus according to claim 1, further comprising a housing with one or more adjustable openings wherein the light emitted from the light emitting diodes exits the apparatus from one or more said adjustable openings.

3. An apparatus according to claim 1, further comprising a filter for blocking one or more predetermined wavelengths emitting from the diodes.

4. An apparatus according to claim 1, further comprising a cover to limit the area of the skin illuminated by the apparatus.

5. An apparatus according to claim 1, further comprising a light intensity adjuster wherein the apparatus is able to provide a full range of low to high light intensities.

6. An apparatus according to claim 1, further comprising a third group of light emitting diodes with a typical peak emission frequency of about 660 nm.

7. An apparatus according to claim 6, further comprising a housing with one or more adjustable openings wherein the light emitted from the light emitting diodes exits the apparatus from one or more said adjustable openings.

8. An apparatus according to claim 6, further comprising a filter for blocking one or more predetermined wavelengths emitting from the diodes.

9. An apparatus according to claim 6, further comprising a cover to limit the area of the skin illuminated by the apparatus.

10. An apparatus according to claim 6, further comprising a light intensity adjuster wherein the apparatus is able to provide a full range of low to high light intensities.

11. A portable lamp apparatus for the treatment of acne, the apparatus comprising:
a plurality of fluorescent tubes, wherein one or more tubes emits a typical peak emission frequency with wavelengths either in the range of 395 nm to 450 nm or in the range of 405 nm to 415 nm, and one or more tubes emits a typical peak emission frequency in the range of 410 nm to 420 nm.

12. A portable lamp apparatus according to claim 11, further comprising a housing with one or more adjustable openings wherein the light emitted from the fluorescent tubes exits the apparatus from one or more said adjustable openings.

13. A portable lamp apparatus according to claim 11, further comprising a filter for blocking one or more predetermined wavelengths emitting from the diodes.

14. A portable lamp apparatus according to claim 11, further comprising a cover to limit the area of the skin illuminated by the apparatus.

15. A portable lamp apparatus according to claim 11, further comprising a light intensity adjuster wherein the apparatus is able to provide a full range of low to high light intensities.

16. A portable lamp apparatus according to claim 11, further comprising one or more fluorescent tubes which emit a typical peak emission frequency of about 660 mn.

17. A portable lamp apparatus according to claim 16, further comprising a housing with one or more adjustable openings wherein the light emitted from the fluorescent tubes exits the apparatus from one or more said adjustable openings.

18. A portable lamp apparatus according to claim 16, further comprising a filter for blocking one or more predetermined wavelengths emitting from the diodes.

19. A portable lamp apparatus according to claim 16, further comprising a cover to limit the area of the skin illuminated by the apparatus.

20. A portable lamp apparatus according to claim 16, further comprising a light intensity adjuster wherein the apparatus is able to provide a full range of low to high light intensities.

21. A method of treating acne, comprising the steps of applying a topical oil solution, used for the treatment of controlling oil, to a portion of skin, removing said solution from the skin, and applying illumination to said portion of skin with an apparatus for the treatment of acne, the apparatus comprising a plurality of light emitting diodes mounted on a substrate, the first group of diodes with either a typical peak emission frequency from wavelengths in the range of 395 nm to 450 nm or a first typical peak emission spectrum in the range of 405 nm to 415 nm and a second emission spectrum substantially in the range of 410 nm to 420 nm;
wherein said plurality of light emitting diodes are operated by an electronic circuit at various frequencies in a forward voltage/current function with electronic and electromechanical components affixed in a controlling circuit and integrated with a common power source.

22. A method of treating acne according to claim 21, wherein the apparatus is further comprised of a housing with one or more adjustable openings wherein the light emitted from the light emitting diodes exits the apparatus from one or more said adjustable openings.

23. A method of treating acne according to claim 21, wherein the apparatus is further comprised of a filter for blocking one or more predetermined wavelengths emitting from the diodes.

24. A method of treating acne according to claim 21, wherein the apparatus is further comprised of a cover to limit the area of the skin illuminated by the apparatus.

25. A method of treating acne according to claim 21, wherein the apparatus is further comprised of a light intensity adjuster wherein the apparatus is able to provide a full range of low to high light intensities.

26. A method of treating acne according to claim 21, wherein the apparatus is further comprised of a third group of light emitting diodes with a typical peak emission frequency of about 660 nm.

27. A method of treating acne according to claim 26, wherein the apparatus is further comprised of a housing with one or more adjustable openings wherein the light emitted from the light emitting diodes exits the apparatus from one or more said adjustable openings.

28. A method of treating acne according to claim 26, wherein the apparatus is further wherein the apparatus is further comprised of a filter for blocking one or more predetermined wavelengths emitting from the diodes.

29. A method of treating acne according to claim 26, wherein the apparatus is further comprised of a cover to limit the area of the skin illuminated by the apparatus.

30. A method of treating acne according to claim 26, wherein the apparatus is further comprised of a light intensity adjuster wherein the apparatus is able to provide a full range of low to high light intensities.

31. A method of treating acne, comprising the steps of applying a topical oil solution, used for the treatment of controlling oil, to a portion of skin, removing said solution from the skin, and applying illumination to said portion of skin with a portable lamp apparatus for the treatment of acne, the portable lamp apparatus comprising a plurality of fluorescent tubes, wherein
one or more tubes emits a typical peak emission frequency with wavelengths either in the range of 395 nm to 450 nm or in the range of 405 nm to 415 nm, and one or more tubes emits a typical peak emission frequency in the range of 410 nm to 420 nm.

32. A method of treating acne according to claim 31, wherein the portable lamp apparatus is further comprised of a housing with one or more adjustable openings wherein the light emitted from the fluorescent tubes exits the apparatus from one or more said adjustable openings.

33. A method of treating acne according to claim 31, wherein the portable lamp apparatus is further comprised of a filter for blocking one or more predetermined wavelengths emitting from the diodes.

34. A method of treating acne according to claim 31, wherein the portable lamp apparatus is further comprised of a cover to limit the area of the skin illuminated by the portable lamp apparatus.

35. A method of treating acne according to claim 31, wherein the portable lamp apparatus is further comprised of a light intensity adjuster wherein the apparatus is able to provide a full range of low to high light intensities.

36. A method of treating acne according to claim 31, wherein the portable lamp apparatus is further comprised of a third group of light emitting diodes with a typical peak emission frequency of about 660 nm.

37. A method of treating acne according to claim 36, wherein the portable lamp apparatus is further comprised of a housing with one or more adjustable openings wherein the light emitted from the fluorescent tubes exits the apparatus from one or more said adjustable openings.

38. A method of treating acne according to claim 36, wherein the portable lamp apparatus is further comprised of a filter for blocking one or more predetermined wavelengths emitting from the diodes.

39. A method of treating acne according to claim 36, wherein the portable lamp apparatus is further comprised of a cover to limit the area of the skin illuminated by the portable lamp apparatus.

40. A method of treating acne according to claim 36, wherein the portable lamp apparatus is further comprised of a light intensity adjuster wherein the apparatus is able to provide a full range of low to high light intensities.
